(19) United States
(12) Patent Application Publication
(10) Pub. No.: US 2012/0052522 A1
(43) Pub. Date: Mar. 1, 2012

(54) Apparatus And Method For The Study Of Metabolism

(75) Inventors: Jose Castro-Perez, New Providence, NJ (US); Robert S. Plumb, Milford, MA (US); Jennifer Harnisch Granger, Salt Lake City, UT (US)

(73) Assignee: WATERS TECHNOLOGIES CORPORATION, Milford, MA (US)

(21) Appl. No.: 13/128,720

(22) PCT Filed: Nov. 17, 2009
(86) PCT No.: PCT/US09/64698

§ 371 Date: Sep. 15, 2011

Related U.S. Application Data
(60) Provisional application No. 61/116,324, filed on Nov. 20, 2008.

(51) Int. Cl. C12Q 1/02; C12M 1/34

(52) U.S. Cl. 435/29; 435/287.1; 435/287.2

(57) ABSTRACT

Embodiments of the present invention feature methods and apparatus in which fluids circulating between a plurality of vessels containing different cell types or tissues are monitored for metabolites following the introduction of a sample.

### CROSS REFERENCE TO RELATED APPLICATION

This applications claims benefit of U.S. Provisional Application No. 61/116,324, filed on Nov. 20, 2008 (Attorney Docket No. W-368-01), the content of which is incorporated herein by reference.

### STATEMENT WITH RESPECT TO FEDERAL SPONSORSHIP

The present invention was made without Federal sponsorship or funds

### FIELD OF THE INVENTION

Embodiments of the present invention are directed to devices for the study of metabolisms and methods for the performance of the study of metabolisms

### BACKGROUND OF THE INVENTION

Metabolics is the science of chemical reactions that occur within cells in a living body which are necessary for life. Due to the processes being inside a living body, the study of these reactions is problematic, for example, the mechanisms in which these reactions occur can be difficult to identify.

Metabolism is the processes involving the chemical reactions that occur within the cells of the living body.

Metabolites are the molecules that are involved in the chemical reactions that occur within a living body. These can include naturally occurring molecules that are produced by the body, molecules that may have been ingested as food, or drugs that have been ingested by the body.

It is useful to understand the processes that occur within living bodies in order to identify important metabolites that may be in healthy subjects, but not within diseased subjects. It is also useful within the pharmaceutical industry to identify or predict the metabolites that are formed in the body when a drug is ingested. This can lead to predicting the stability and effectiveness of the metabolites that are formed.

Current techniques of finding metabolites include using liver microsomes, liver slices and hepatocytes or perfused livers. However, whilst these techniques can give some useful information, it is not predictive of in-vivo metabolisms.

It is therefore desired for an apparatus and method for identifying or studying in vivo metabolisms in a way that overcomes the difficulties of the apparatus and methods described in the prior art.

As used herein, the term in vivo means within a living organism.

### SUMMARY OF THE INVENTION

Embodiments of the present invention are directed to apparatus and methods for the study of metabolism and their associated metabolites

One embodiment of the present invention is directed to an apparatus having the following major elements: a first containment means, a second containment means, conduit means, extraction means and analysis means. The first containment means has at least one first cell held in an environment suitable for sustaining metabolic functions of the first cell. The first cell is for receiving a sample comprising at least one compound and exhibiting at least one first cell response. The first cell response potentially comprises forming a first cell metabolite, cell death, cell growth, cell differentiation, teratogenesis, mutagenesis, contractions, secretions, RNA production, protein and peptide synthesis and the like.

The second containment means has at least one second cell held in an environment suitable for sustaining metabolic functions of the second cell. The second cell is for receiving the sample and the first cell metabolite, if formed and exhibiting at least one second cell response. The second cell response potentially comprises forming a second cell metabolite, and any of the first cell responses.

The conduit means is in fluid communication with the first containment means and the second containment means for circulating fluid comprising sample and, if present, first cell metabolites and second cell metabolites between said first containment means and said second containment means. The conduit means allows the first cell and said second cell to exhibit at least one cell response following the introduction of a sample.

The extraction means is in communication with at least one of said conduit means, first containment means and second containment means for removing an aliquot of the fluid.

The analysis means in fluid communication with the extraction means for detecting the presence of said at least one of the first cell metabolite and said second cell metabolite, if formed. The analysis means allows the first cell and second cell to be monitored over time for a response to a sample.

As used herein, the term first containment means and second containment means refers to closed or substantially closed vessels or biological reaction vessels commonly used in the art of cell culture and tissue and organ studies to sustain such cell cultures, tissues and organs for a period of time.

As used herein, the term conduit means refers to piping, tubing, capillaries, hoses and other devices and apparatus to transport fluids. As used herein, the term further comprises pumps and valves for propelling such fluids. The term “fluid communication” means plumbed together to allow the passage of fluids.

As used herein, the term extraction means refers to devices for removing an aliquot of fluid in a continuous or periodic way. For example, without limitation, the extraction means may comprise a port with a needle, which needle is placed in communication with the port at selected times. This port may have dual functions to remove fluid or place sample into the conduit means. In the alternative, the extraction means may comprise a shunt, or branch of piping, tubing, capillary, hose and the like for removing an aliquot of the fluid.

As used herein, the term analysis means refers to any device for analysing the fluid for the presence of metabolites, hormones, cellular secretions and the like. Such devices comprise, by way of example, without limitation mass detectors or spectrometers, photo-detectors, electro magnetic detectors, and the like. In order to ascertain cellular responses other than of a chemical nature, the first containment vessel and the second containment vessel may comprise or be placed in communication with or have means for extracting selected cells. Selected cells can be withdrawn through withdrawal ports and/or conduits. Or, the containment vessels can be equipped with microscopes or other imaging devices.

Preferably, the apparatus further comprises control means in signal communication with the analysis means. And, even more preferably, the control means is in signal communication with extraction means and conduit means to control the movement of fluids, the extraction of aliquots and the analysis means to determine the presence or absence of a first metabolite and/or second metabolite. As used herein, the term “signal communication” refers to the exchange of electro-magnetic or photo or acoustic signals by means of wires, radio or photo transmission and the like. The term “control means” refers to computational devices such as computer processing units (CPUs) known in the art and commonly incorporated in personal and mainframe computers and servers, instrument control devices, or embedded in the instruments such as chromatographic systems and detectors.

The apparatus preferably is able to extract aliquots from the first containment means and the second containment means of conduits associated with the flow of fluid from each to detect the formation of at least one metabolite originating with the first cell and/or second cell. Where both the first cell and second cell produce metabolites, such formation of metabolites can be related to the passage of fluid, such as a first pass effect, or second pass effect, over time. The formation of metabolites can be related to the first cell or the second cell and be dependent on the prior formation of a metabolite. Thus, complex metabolic pathways can be analysed with as many containment means as desired, with as many cell types as desired. The cell response can be the formation of a plurality of metabolites selected from the group of first cell metabolites and second cell metabolites. These first and second cell metabolites may each be further metabolised.

Preferably, the control means monitors the circulation times and records the number of passes made to each first containment means and second containment means and each subsequent containment means, to associate one or more metabolites with a first cell or a second cell or a subsequent cell type.

Preferably, the sample is introduced through a sample opening in communication with at least one of the conduit means, first containment means and second containment means. In the alternative, the apparatus can have the sample present in the fluid upon start up.

Preferably, the first cell and the second cell are selected from the group of cell types associated with an organ and flora. For example, the organ may be a liver and the cells hepatocytes. By way of further example, without limitation, the cell type may be stem cells for which a differentiation is desired. Or, the cells may be selected in a manner to retain structural relationships to evaluate clearance such as cells from kidney or the gut. The cells may further comprise a microbial system includes gut microflora. Preferred cells and microflora are derived from human sources to allow human metabolic processed to be studied.

A further embodiment of the present invention is further directed to a method for determining a cellular response to an application of a sample. The method comprises the steps of providing an apparatus having a first containment means, second containment means, conduit means, extraction means and analysis means. The first containment means has at least one first cell held in an environment suitable for sustaining metabolic functions of said first cell. The first cell is for receiving a sample comprising at least one compound and exhibiting at least one cell response which cell response potentially comprises forming a first cell metabolite. The second containment means has at least one second cell held in an environment suitable for sustaining metabolic functions of the second cell. The second cell is for receiving the sample and exhibiting at least one cell response which cell response potentially comprises forming a second cell metabolite. The conduit means is in fluid communication with the first containment means and the second containment means for circulating fluid comprising sample and, if present, first cell metabolites and second cell metabolites between the first containment means and the second containment means. The conduit means allows the first cell and said second cell to exhibit at least one cell response following the introduction of a sample. The extraction means is in communication with at least one of the conduit means, first containment means and second containment means for removing an aliquot of the fluid. The analysis means is in fluid communication with the extraction means for detecting the presence of said at least one of said first cell metabolite and the second cell metabolite, if formed, to allow the first cell and second cell to be monitored over time for a response to the sample. The method further comprises the step of placing a sample in at least one of the conduit means, first containment means and second containment means and extracting an aliquot of fluid from extraction means and analysing the aliquot for the presence of one or more first metabolites and second metabolites indicative of a cellular response.

These and other features and advantages will be apparent to those skilled in the art upon viewing the drawing depicting embodiments of the invention and studying the detailed description that follows.

### BRIEF DESCRIPTION OF THE DRAWINGS

FIG. 1 depicts an apparatus in accordance with the invention

### DETAILED DESCRIPTION OF THE DRAWINGS

Embodiments of the invention will be described with respect to the study of metabolic pathways with the understanding that the invention has broad application in other fields as well.

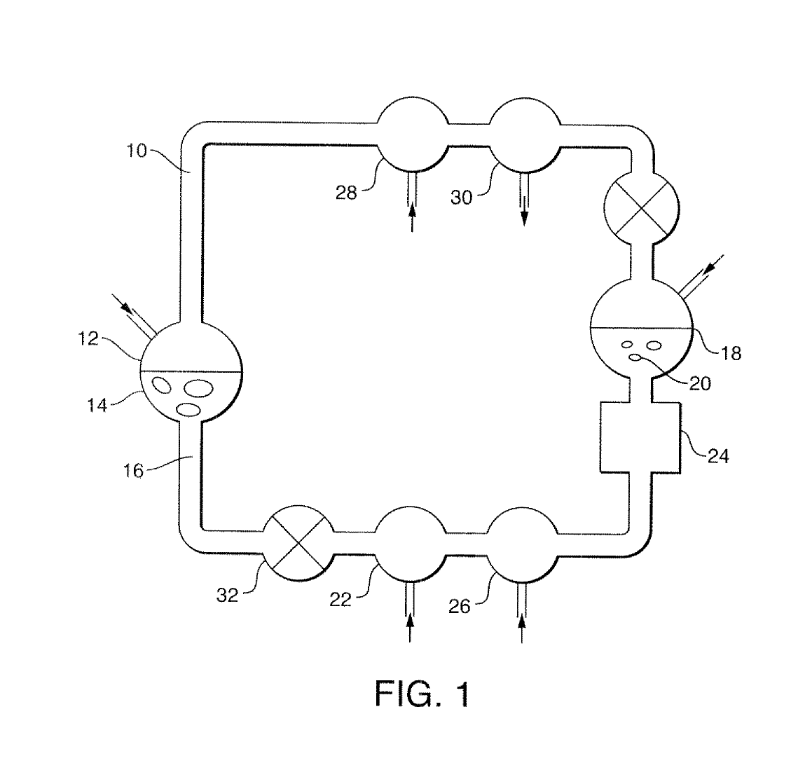

Turning now to FIG. 1, such figure depicts an apparatus, generally designated by the numeral 10, in accordance with the preferred embodiment of the invention.

The apparatus comprises a first containment means (12) having at least one first cell held in an environment suitable for sustaining metabolic functions of the first cell (14). The first cell (14) is for receiving a sample comprising at least one compound and exhibiting at least one first cell response. The first cell response potentially comprises forming a first cell metabolite, cell death, cell growth, cell differentiation, teratogenesis, mutagenesis, contractions, secretions, RNA production, protein and peptide synthesis and the like.

In a preferred embodiment the first containment means is made of fused silica, glass, plastic or metal. Preferred plastics are selected from one or more thermoplastics currently available as exemplified in the text Modern Plastics Handbook, Charles A Harper, editor; McGraw-Hill (2005). Preferred plastics include polyethylrethylketone, sold under the trademark PEEK™ (Dupont), Polyfluoroalkyl polymers sold under the trademark TEFLON® (Dupont) and PTFE, polyimide polymers, polyamide imide polymers, polyethylene polymers, polyvinylindene fluoride polymers, polychlorofluoroalkyl polymers, known in the trade as PCTFE.

The first cell may be an organ, or cells from an organ involved in metabolic systems. The organ may be a liver, liver microsomes, spheroid complexes, a kidney, cells from a kidney, any other organ in which metabolic reactions occur, or cells from any other organ in which metabolic reactions occur. In one embodiment the organ or organ cells are human cells. In a less preferred embodiment the organ cells are from an animal.

The second containment means (18) has at least one second cell (20) held in an environment suitable for sustaining metabolic functions of the second cell. The second cell (20) is for receiving the sample and the first cell metabolite, if formed and exhibiting at least one second cell response. The second cell response potentially comprises forming a second cell metabolite, and any of the first cell responses.

In a preferred embodiment the second containment means is made of fused silica, glass, plastic or metal. Preferred plastics are selected from one or more thermoplastics currently available as exemplified in the text Modern Plastics Handbook, Charles A Harper, editor; McGraw-Hill (2005). Preferred plastics comprise polyethylethylketone, sold under the trademark PEEK™ (Dupont), Polyfluoroalkyl polymers sold under the trademark TEFLON® (Dupont) and PTFE, polyimide polymers, polyamide imide polymers, polyethylene polymers, polyvinylindene fluoride polymers, polychlorofluoroalkyl polymers, known in the trade as PCTFE.

The second cell may be gut microflora, or microflora from any other communities. The cells may be any further cells in a metabolic system. In one embodiment the cells are human cells. In a less preferred embodiment the cells are from an animal.

The first containment means and second containment means may be closed or substantially closed vessels or biological reaction vessels commonly used in the art of cell culture and tissue and organ studies to sustain such cell cultures, tissues and organs for a period of time.

The conduit means (16) is in fluid communication with the first containment means (12) and the second containment means (18) for circulating fluid comprising sample and, if present, first cell metabolites and second cell metabolites between said first containment means (12) and said second containment means (18). The conduit means (16) allows the first cell and said second cell to exhibit at least one cell response following the introduction of a sample.

The conduit means may be piping, tubing, capillaries, hoses and other devices and apparatus for the transport fluids. As used herein, the term further comprises pumps and valves for propelling such fluids.

The conduit means may be made of fused silica, glass, plastic or metal. Preferred plastics are selected from one or more thermoplastics currently available as exemplified in the text Modern Plastics Handbook, Charles A Harper, editor; McGraw-Hill (2005). Preferred plastics comprise polyethylethylketone, sold under the trademark PEEK™ (Dupont), Polyfluoroalkyl polymers sold under the trademark TEFLON® (Dupont) and PTFE, polyimide polymers, polyamide imide polymers, polyethylene polymers, polyvinylindene fluoride polymers, polychlorofluoroalkyl polymers, known in the trade as PCTFE. The conduit means circulates the sample around the apparatus.

Fluid communication refers to parts of the apparatus being plumbed together to allow the passage of fluids.

The extraction means (22) is in communication with at least one of said conduit means (16), first containment means (12) and second containment means (18) for removing an aliquot of the fluid.

The extraction means refers to devices for removing an aliquot of fluid in a continuous or periodic way. For example, without limitation, the extraction means may comprise a port with a needle, which needle is placed in communication with the port at selected times. This port may have dual functions to remove fluid or place sample into the conduit means. In the alternative, the extraction means may comprise a shunt, or branch of piping, tubing, capillary, hose and the like for removing an aliquot of the fluid. The extraction means may further be any sample extraction pump, or any method of extracting a sample from an apparatus. The extraction means may further come from the group comprising a looped extraction system, a taped extraction system, a flow splitter or diverter, or a syringe and suitable resealing mechanism. Other examples of extraction means would be known to the skilled person.

The analysis means (not shown) is in fluid communication with the extraction means (22) for detecting the presence of said at least one of the first cell metabolite and said second cell metabolite, if formed. The analysis means allows the first cell (14) and second cell (20) to be monitored over time for a response to a sample.

The analysis means may be any device for analysing the fluid for the presence of metabolites, hormones, cellular secretions and the like. Such devices comprise, by way of example, without limitation mass detectors or spectrometers, photo-detectors, electro magnetic detectors, and the like.

Preferably the analysis means is a mass spectrometer, a LCMS system, a GCMS system, an ion mobility spectrometer, a Raman spectrometer, an IR spectrometer, a NMR spectrometer, or any other type of analytical instrument. Most preferably the analysis means is an Acquity UPLC liquid Chromatograph followed by a SYNAPT HDMS mass spectrometer available from Waters Corporation, Milford, USA. By the analysis of the samples as they circulate around the system, it is possible to identify the species that are present by analytical techniques which are well known to the skilled person. These techniques may be aided by software. Preferably the software is Masslynx available from Waters Corporation, Milford, USA.

The apparatus may further comprise control means in signal communication with the analysis means. And, even more preferably, the control means is in signal communication with extraction means and conduit means to control the movement of fluids, the extraction of aliquots and the analysis means to determine the presence or absence of a first metabolite and/or second metabolite. Control means may be computational devices such as computer processing units (CPUs) known in the art and commonly incorporated in personal and mainframe computers and servers, instrument control devices, or embedded in the instruments such as chromatographic systems and detectors.

Signal communication refers to the exchange of electro-magnetic or photo or acoustic signals by means of wires, radio or photo transmission and the like to control the operation of the whole of, or any part of the apparatus.

In one embodiment the first chamber is flushed with Oxygen gas. The second chamber is flushed with Nitrogen gas. Between the first chamber and the second chamber is a vacuum degasser (24). The vacuum degasser is arranged in between the first confinement chamber and the second confinement chamber. The vacuum degasser is arranged to remove the oxygen from the sample flow into the second chamber. Oxygen getting in to the second chamber would destroy the anaerobic environment in the second chamber and may kill the microbes.

The apparatus further comprises at least one injection means (26). The injection means may be any sort of apparatus suitable for injecting a sample of interest into the conduit means. This may be any sample injection or preparation system, examples of suitable systems include a looped flow injection system coupled to a switching valve, a pressurised mixing tee or a pumped injecting syringe. Other examples of injection means would be known to the skilled person.

In one embodiment a drug metabolite is injected by the injection means (26) into the conduit means (16). The sample passes through the vacuum degasser (24) and into the second containment means (18) where the sample may react with the at least one second cell (20). The sample passes around the conduit means (16) from the second containment means (18) to the first containment means (12). The sample may then react with the at least one first cell (14). The sample may then pass around the conduit means back through the vacuum degasser, and circulate around the apparatus for as many laps as necessary. The reactions that occur as the sample circulates around the system may be monitored by extraction of a small quantity of sample from the extraction means as the sample circulates around the system. In the preferred embodiment a second injection means (28) and a second extraction means (30) are provided so as to enable the analysis of samples between the second cells and the first cells, and to monitor if different reactions occur dependent upon the different order the sample encounters the first and second cells.

In the preferred embodiment, there is at least one propulsion means (32) to propel the sample around the system. In the embodiment of FIG. 1 there are two propulsion means, but it would be apparent to a person skilled in the art that there may be any number of propulsion means. The propulsion means may be any means of creating a pressure differential across parts of the system, for example a pump. In the preferred embodiment the apparatus further contains a second pump (34) to further propel sample around the system

A person skilled in the art would appreciate that the system may have any number of further containment means for further cells which may react with the sample. In this event further injection means, extraction means and propulsion means may be provided within the system.

The apparatus preferably is able to extract aliquots from the first containment means and the second containment means of conduits associated with the flow of fluid from each to detect the formation of at least one metabolite originating with the first cell and/or second cell. In order to ascertain cellular responses other than of a chemical nature, the first containment vessel and the second containment vessel may comprise or be placed in communication with or have means for extracting selected cells. Selected cells can be withdrawn through withdrawal ports and/or conduits. Or, the containment vessels can be equipped with microscopes or other imaging devices.

Where both the first cell and second cell produce metabolites, such formation of metabolites can be related to the passage of fluid, such as a first pass effect, or second pass effect, over time. The formation of metabolites can be related to the first cell or the second cell and be dependent on the prior formation of a metabolite. Thus, complex metabolic pathways can be analysed with as many containment means as desired, with as many cell types as desired. The cell response can be the formation of a plurality of metabolites selected from the group of first cell metabolites and second cell metabolites. These first and second cell metabolites may each be further metabolised.

In one embodiment the control means monitors the circulation times and records the number of passes made to each first containment means and second containment means and each subsequent containment means, to associate one or more metabolites with a first cell or a second cell or a subsequent cell type.

In one embodiment of the invention, the sample is introduced through a sample opening in communication with at least one of the conduit means, first containment means and second containment means. In the alternative, the apparatus can have the sample present in the fluid upon start up.

In the preferred embodiment, the first cell and the second cell are selected from the group of cell types associated with an organ and flora. For example, the organ may be a liver and the cells hepatocytes. By way of further example, without limitation, the cell type may be stem cells for which a differentiation is desired. Or, the cells may be selected in a manner to retain structural relationships to evaluate clearance such as cells from kidney or the gut. The cells may further comprise a microbial system includes gut microflora. Preferred cells and microflora are derived from human sources to allow human metabolic processed to be studied.

A further embodiment of the present invention is further directed to a method for determining a cellular response to an application of a sample. The method comprises the steps of providing an apparatus having a first containment means, second containment means conduit means, extraction means and analysis means. The first containment means has at least one first cell held in an environment suitable for sustaining metabolic functions of said first cell. The first cell is for receiving a sample comprising at least one compound and exhibiting at least one cell response which cell response potentially comprises forming a first cell metabolite. The second containment means has at least one second cell held in an environment suitable for sustaining metabolic functions of the second cell. The second cell is for receiving the sample and exhibiting at least one cell response which cell response potentially comprises forming a second cell metabolite. The conduit means is in fluid communication with the first containment means and the second containment means for circulating fluid comprising sample and, if present, first cell metabolites and second cell metabolites between the first containment means and the second containment means. The conduit means allows the first cell and said second cell to exhibit at least one cell response following the introduction of a sample. The extraction means is in communication with at least one of the conduit means, first containment means and second containment means for removing an aliquot of the fluid. The analysis means is in fluid communication with the extraction means for detecting the presence of said at least one of said first cell metabolite and the second cell metabolite, if formed, to allow the first cell and second cell to be monitored over time for response to the sample. The method further comprises the step of placing a sample in at least one of the conduit means, first containment means and second containment means and extracting an aliquot of fluid from extraction means and analysing the aliquot for the presence of one or more first metabolites and second metabolites indicative of a cellular response.

The apparatus may be used for the monitoring of up and down regulation of metabolites through the application of competing metabolite substrates and other potential interfering substances.

This apparatus may also be suitable for the monitoring and analysis of any other biological processes that may occur outside the body. An example of this is the fermentation process.

The apparatus can be adapted to allow the conditions in the different parts of the apparatus to be varied so as to suit the preferred environment of the first cells and second cells.

The advantages of the invention will be apparent to a person skilled in the art. Therefore, the present invention should not be limited to the details described in the description but should encompass such subject matter as defined in the claims.

1. An apparatus for the study of metabolism comprising:
a. a first containment means having at least one first cell held in an environment suitable for sustaining metabolic functions of said first cell, said first cell for receiving a sample comprising at least one compound and said first cell exhibiting at least one first cell response which first cell response potentially comprises forming a first cell metabolite;
b. a second containment means having at least one second cell held in an environment suitable for sustaining metabolic functions of the second cell said second cell for receiving said sample and said first cell metabolite and exhibiting at least one second cell response which second cell response potentially comprises forming a second cell metabolite;
c. conduit means in fluid communication with said first containment means and said second containment means for circulating fluid comprising sample and, if present, first cell metabolites and second cell metabolites between said first containment means and said second containment means to allow said first cell and said second cell to exhibit at least one first cell response or second cell response following the introduction of a sample;
d. extraction means in fluid communication with at least one of said conduit means, first containment means and second containment means for removing an aliquot of said fluid; and,
e. analysis means in fluid communication with said extraction means for detecting the presence of said at least one of said first cell metabolite and said second cell metabolite, if formed, to allow said first cell and second cell to be monitored over time for a first cell response or a second cell response to a sample.

2. The apparatus of claim 1 wherein said cell response further comprises at least one of the group of cell death, cell viability, cell growth, cell teratogenesis, cell mutagenesis, cell contraction, cell RNA, cellular protein or peptide production.

3. The apparatus of claim 1 wherein said apparatus further comprises control means in signal communication with said analysis means, said control means receiving one or more signals representative of a cell response.

4. The apparatus of claim 3 wherein at least one of said first cell response and second cell response is the formation of at least one metabolite.

5. The apparatus of claim 3 wherein said cell response is the formation of at least one first cell metabolite.

6. The apparatus of claim 4 wherein said metabolite is at least one second cell metabolite.

7. The apparatus of claim 3 wherein said cell response is the formation of a plurality of metabolites selected from the group of first cell metabolites and second cell metabolites.

8. The apparatus of claim 1 further comprising control means said control means monitoring circulation times and recording the number of passes made to each first containment means and second containment means, to associate one or more metabolites with a first cell or a second cell.

9. The apparatus as claimed in claim 1 wherein said first containment means holds cells associated with a first organ type.

10. Apparatus as claimed in claim 9 wherein said organ type is a liver.

11. Apparatus as claimed in claim 9 wherein said organ is a human organ.

12. Apparatus as claimed in claim 1 where said second containment means holds a microbial system.

13. Apparatus as claimed in claim 12 where said microbial system includes gut microflora.

14. Apparatus as claimed in claim 12 wherein said microbial system is a human microbial system.

15. Apparatus as claimed in claim 1 wherein said analysis means includes at least one mass spectrometer.

16. Apparatus as claimed in claim 1 wherein control means monitors said analysis means for at least one of the group of first metabolites and second metabolites.

17. A method for determining a cellular response to an application of a sample comprising:
a. Providing an apparatus having a first containment means, second containment means conduit means, extraction means and analysis means, said first containment means having at least one first cell held in an environment suitable for sustaining metabolic functions of said first cell, said first cell for receiving a sample comprising at least one compound and said first cell exhibiting at least one cell response which cell response potentially comprises forming a first cell metabolite; said second containment means having at least one second cell held in an environment suitable for sustaining metabolic functions of the second cell said second cell for receiving said sample and said first cell metabolite and exhibiting at least one cell response which cell response potentially comprises forming a second cell metabolite; said conduit means in fluid communication with said first containment means and said second containment means for circulating fluid comprising sample and, if present, first cell metabolites and second cell metabolites between said first containment means and said second containment means to allow said first cell and said second cell to exhibit at least one cell response following the introduction of a sample; said extraction means in fluid communication with at least one of said conduit means, first containment means and second containment means for removing an aliquot of said fluid; and, said analysis means in fluid communication with said extraction means for detecting the presence of said at least one of said first cell metabolite and said second cell metabolite, if formed, to allow said first cell and second cell to be monitored over time for a response to a sample;
b. placing a sample in at least one of said conduit means, first containment means and second containment means and extraction an aliquot of fluid from extraction means and analysing said aliquot for the presence of one or more first metabolites and second metabolites indicative of a cellular response.

18. Method as claimed in claim 17 wherein said at least one first cell comprises cells from a selected organ type.

19. Method as claimed in claim 18 wherein said organ type is a liver.

20. Method as claimed in claim 17 wherein said cells are of human organ.

21. Method as claimed in claim 17 where said at least one second cell is derived from a microbial system.

22. Method as claimed in claim 21 where said microbial system includes gut microflora.

23. Method as claimed in claim 22 wherein said microbial system is a human microbial system.

24. Method as claimed in claim 17 wherein said analysis means includes at least one mass spectrometer.
